(19) United States
(12) Patent Application Publication
(10) Pub. No.: US 2013/0216019 A1
(43) Pub. Date: Aug. 22, 2013

(54) X-RAY CT APPARATUS AND MEDICAL IMAGE DISPLAY METHOD

(71) Applicants: Kabushiki Kaisha Toshiba, (US); Toshiba Medical Systems Corporation, (US)

(72) Inventors: Tatsuo MAEDA, Nasushiobara-shi (JP); Yoshihiro IKEDA, Sakura-shi (JP)

(73) Assignees: Toshiba Medical Systems Corporation, Otawara-shi (JP); Kabushiki Kaisha Toshiba, Tokyo (JP)

(21) Appl. No.: 13/772,798

(22) Filed: Feb. 21, 2013

(30) Foreign Application Priority Data
Feb. 22, 2012 (JP) 2012-036161

(51) Int. Cl. A61B 6/00; A61B 6/03

(52) U.S. Cl. CPC A61B 6/484; A61B 6/035
USPC 378/19

(57) ABSTRACT

The collecting part of the X-ray CT apparatus according to the embodiment scans a subject using X-rays and collects data. The memory prerecords a basic image including the designated area of the subject. The controller instructs the collector to repeatedly perform the first scan, using the conditions for the first scan, of the designated area of the subject to who contrast agent has been administered. The controller then receives the specified trigger input, and instructs the second scan of the subject using the conditions for the second scan. The image generator repeatedly produces the first medical image based on the data collected when the first scan is being performed, and produces a subtraction image from the first medical image and the basic image. The image generator then produces the second medical image based on the data collected when the second scan is being performed.

### CROSS-REFERENCE TO RELATED APPLICATION

This application is based upon and claims the benefit of priority from Japanese Patent Application No. 2012-036161, filed Feb. 22, 2012; the entire contents of which are incorporated herein by reference.

### FIELD

The embodiment of the present invention relates to an X-ray CT apparatus and a medical image display method.

### BACKGROUND

X-ray CT (Computed Tomography) apparatus uses X-rays to scan a subject, and processes the data collected using a computer. In doing so, X-ray CT apparatuses produce medical images of the inside of the subject.

Specifically, X-ray CT apparatuses irradiate multiple times X-rays to the subject from varying directions, use X-ray detector to detect the X-rays that have passed through the subject, and collect multiple of detecting data. The collected detecting data is subjected to A/D conversion by the data collector. Subsequently, the A/D converted data is sent to a console apparatus. The console apparatus pre-processes said detecting data and produces projection data. Subsequently the console apparatus performs reconstructing based on the projection data. The X-ray CT apparatus produces an X-ray CT medical image based on this reconstructed data.

X-ray CT medical images play a valuable role in a wide range of medical activities, including diagnostics, treatment, operation planning, etc. X-ray CT apparatuses also make it possible to reconstruct medical images in real time while capturing scanning them. By reconstructing X-ray CT medical images in real time, X-ray CT apparatuses enable X-ray CT medical images to be displayed at almost the same time as they are being captured (in real time).

In some cases, scanning is done by X-ray CT apparatuses using a contrast agent. When the scanning using a contrast agent is done, the contrast agent is introduced into the designated area (specified internal organs, etc.) of the subject by injecting contrast agent for the subject. When scanning is done with the contrast agent introduced into the designated area, it is possible to highlight the specified area through contrast difference. Highlighting allows the provision of medical images to the user in which the specified area can be easily identified. Scanning using contrast agent contributes to, for example, the diagnosis of the presence or otherwise of neoplasm in the specified area. Scanning using contrast agent does, however, require consideration of the time lapse between injecting the contrast agent into the subject and its arrival at the designated area. In other words, it is necessary to avoid completing the scanning prior to the contrast agent arriving in the designated area. Furthermore, it is also necessary to avoid a situation wherein scanning begins only after the contrast agent has passed through the designated area.

In order to solve the problem above, a scanning method exists that performs SURE Start scans (prep scans, monitoring scans or pre-scans). A SURE Start scan involves scanning to observe the entry of the contrast agent into the vicinity of the region of interest, prior to the scanning of the region of interest including the specified area (hereinafter referred to, in places, as the “main scan”). If, for example, the contrast agent is introduced to the area scanned for the SURE Start scan, in other words, the area upstream of the region of interest, the area impacted by the contrast agent will be highlighted in the X-ray CT medical images obtained from the SURE Start scan. By meeting specified conditions based on this contrast image, it is possible to predict when the contrast agent will flow the region of interest. At the point at which the X-ray CT apparatus needs to switch over from performing the SURE Start scan to the main scan, it moves on to the main scan.

An example of methods used to ensure the timing of switching from the SURE Start scan to the main scan is controlling the X-ray CT apparatus based, for example, on a TDC (Time Density Curve). A TDC is a curve that expresses changes in the density of the contrast agent over time in the specified observation position while the SURE Start scan performs.

Since the contrast agent does not flow the region of interest directly after the subject is injected with it, there is no change in the CT number within the region of interest. When the contrast agent flows the region of interest, however, the CT number changes (increases). The X-ray CT apparatus records a specified threshold, and at the point at which the CT number in the observation location is equal to or exceeds the threshold, it moves to the main scan. In other words, at the point at which the CT number in the TDC is reached or exceeded, it is assumed that the contrast agent has flowed the region of interest, and the main scan begins.

The X-ray CT apparatus moving from the SURE Start scan to the main scan allows the flow of the contrast agent to the region of interest, captured by the main scan, to be displayed in real time. In other words, it is possible to highlight in real time the region of interest in the medical images captured by the main scan using contrast agent.

### BRIEF DESCRIPTION OF THE DRAWINGS

FIG. 1 is a block diagram of the X-ray CT apparatus according to Embodiment 1.

FIG. 2 is a flow chart showing an outline of the operation of the X-ray CT apparatus according to Embodiment 1.

FIG. 3 is a flow chart showing an outline of the operation of the X-ray CT apparatus according to Embodiment 1.

FIG. 4 is a block diagram of the X-ray CT apparatus according to Embodiment 2.

FIG. 5 is a flow chart showing an outline of the operation of the X-ray CT apparatus according to Embodiment 2.

FIG. 6 is a flow chart showing an outline of the operation of the X-ray CT apparatus according to Embodiment 2.

FIG. 7 is a flow chart showing an outline of the operation of the X-ray CT apparatus according to Embodiment 2.

### DETAILED DESCRIPTION

The X-ray CT apparatus according to the embodiment comprises a collector, a memory, a controller and an image generator. The collector scans the subject with X-rays and collects data. The memory prerecords a basic image of the subject, including the designated area. The controller controls the collector, instructing it to repeatedly perform the first scan of the designated area of the subject subsequent to the administration of contrast agent, under the conditions for the first scan. The controller then receives the specified trigger input, and instructs the second scan of the subject, under the conditions for the second scan. The image generator repeatedly produces the first medical image, based on the data collected while the first scan is being performed. The image generator then produces a subtraction image based on the first medical image and the basic image. Next, the image generator produces the second medical image, based on the data collected while the second scan is being performed.

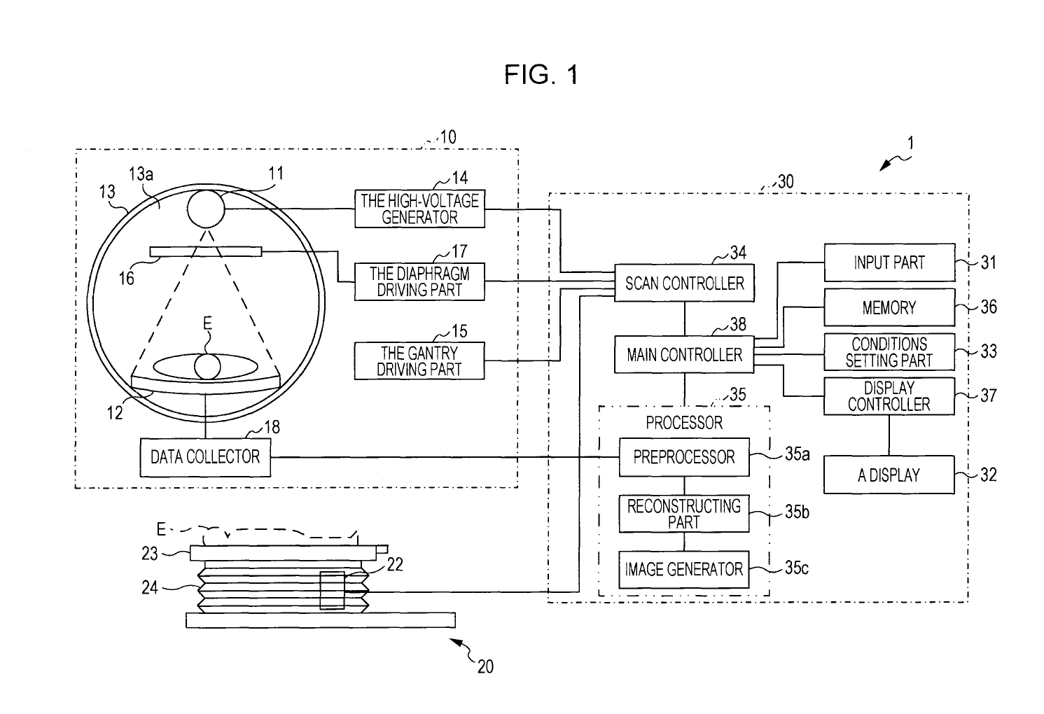

The following is an explanation in reference to FIGS. 1 through 7 of the X-ray CT apparatus according to the embodiment.

### First Embodiment

The following is an explanation of the structure of the X-ray CT apparatus 1 according to Embodiment 1, in reference to FIGS. 1 through 3. Since “medical image” and “medical image data” corresponds on one-to-one relationship effectively, in this embodiment they are the same thing, in some cases, used as though equal.

### (Apparatus Structure)

As shown in FIG. 1, the X-ray CT apparatus 1 comprises a gantry device 10, a bed apparatus 20, and console apparatus 30.

### (Gantry Equipment)

The gantry device 10 is a device that irradiates X-rays onto the subject E and collects the detected data from these X-rays transmitted through the subject E. The gantry device 10 comprises an X-ray generating part 11, an X-ray detector 12, a rotational body 13, a high voltage generating part 14, a gantry driving part 15, an X-ray diaphragm part 16, a diaphragm driving part 17, and a data collector 18.

The X-ray generating part 11 includes an X-ray tube (not shown), which generates X-rays. The X-ray tube is a vacuum tube that generates conical or pyramidal beams. The subject E is exposed (irradiated) to the generated X-rays. The X-ray detector 12 includes multiple X-ray detection elements (not shown). The X-ray detector 12 detects X-ray intensity distribution data (hereinafter, sometimes referred to as “detection data”) for the X-rays passing through the subject E using the X-ray detection elements. Furthermore, the X-ray detector outputs the detection data as a current signal. A 2-dimensional X-ray detector (plane detector) which has multiple detection elements located in each of two orthogonal directions (the slice direction and the channel direction) may be used as the X-ray detector 12. In this structure, multiple X-ray detection elements are provided, for example, in 320 rows along the slice direction. Thereby, using the X-ray detectors in a plurality of rows, by scanning with one revolution, a three-dimensional medical imaging region having a width in the slice direction can be scanned (a volume scan). The slice direction is equivalent to the axial direction on the body of the subject E, while the channel direction is equivalent to the rotation direction of the X-ray generating part 11. In this embodiment, however, there is no need to use an X-ray detector of the type described above, with multiple rows.

The rotational body 13 is a member that supports the X-ray generating parts 11 and the X-ray detector 12 such that they are opposed across the subject E. The rotational body 13 has an opening 13a penetrating therethrough in the slice direction. In the gantry device 10, the rotational body 13 is arranged such that it rotates in a circular orbit around the subject E.

The high-voltage generator 14 applies a high voltage to the X-ray generating part 11. The X-ray generating part 11 generates X-rays based on this high voltage. The gantry driving part 15 drives the rotation of the rotational body 13. The X-ray diaphragm part 16 has a slit (opening) of a specified width, the alteration of which allows the adjustment of the X-ray fan angle (the angle in which the rays spread in the channel direction) and the X-ray cone angle (the angle in which they spread in the slice direction) of X-rays to which the subject is exposed from the X-ray generating part 11. The diaphragm driving part 17 drives the X-ray diaphragm part 16 so that the X-rays generated by the X-ray generating part 11 are the specified shape.

The data collector 18 (DAS: Data Acquisition System) collects detected data from the X-ray detector 12 (each X-ray detection element). Additionally, the data collector 18 converts the collected detection data (current signal) into a voltage signal. The data collector 18 also periodically integrates and amplifies this voltage signal, before converting it into a digital signal. The data collector 18 then sends the detection data that has been converted into a digital signal to the console apparatus 30 (processor 35 (see below)). The reconstructing part 35b (see below) may be structured so as to obtain CT medical images in real time, by quickly performing reconstruction based on the detection data collected by the data collector 18.

### (Bed Apparatus)

The bed apparatus 20 is used to hold and move the subject E to be scanned. The bed apparatus 20 includes a bed apparatus 20 and a bed operating part 22. The bed apparatus 20 includes a bed board 23 to hold the subject E, and a base 24 to hold the bed board 23. The bed board 23 can be moved by the bed operating part 22 both axially to the body of the subject E and orthogonally in the axial direction of the body of the subject E. In other words, the bed operating part 22 can insert and remove the bed board 23 on which the subject is held to and from the opening 13a of the rotational body 13. The base 24 can move the bed board 23 up and down (orthogonally in the axial direction of the body of the subject E) using the bed operating part. The bed apparatus 20 may be used without the bed board 23. In other words, a structure that involves moving the gantry device 10 in relation to the bed apparatus 20 is also included in the present embodiment of the X-ray CT apparatus.

### (Console Apparatus)

The console apparatus 30 is used to input instructions to the X-ray CT apparatus. In addition, the console apparatus 30 also functions to reconstruct CT medical image data (tomographic image data and volume data) showing the internal state of the subject E from detected data collected by the gantry device 10. The console apparatus 30 comprises an input part 31, a display 32, and conditions setting part 33, a scan controller 34, a processor 35, a memory 36, a display controller 37, and a main controller 38.

### <Input Part>

The input part 31 is used as the input device from which various operations are performed with regard to the console apparatus 30. The input part 31 comprises, for example, a keyboard, a mouse, a trackball, a joystick, etc. Alternatively, the GUI (Graphical User Interface) displayed on the display 32 can also be used as the input part 31.

The input part 31 allows the user (operator) of the X-ray CT apparatus 1 to operate the apparatus or input various instructions at will. For example, the input part 31 can be used in optional operations of the gantry device 10 relating to scanning. Such optional operations relating to scanning can include operating instructions to start or stop the SURE Start scan or main scan. In addition, the input part 31 can be used to the operating instructions regarding to start or stop intermittent scanning. Furthermore, the input part 31 can also be used for the setting of various parameters used by the X-ray CT apparatus 1, such as the defining of scanning conditions. Additionally, the input part 31 can be used for the setting of various parameters used in reconstructing by the reconstructing part 35b. The input part 31 is also used in defining the WL (window level) and WW (window width) of the X-ray CT medical images. The input part 31 is also used in the manual operation of the bed board 23, and in the vocal output of instructions to the subject to breathe, etc. The input part 31 is also used for the overall maintenance etc. of the X-ray CT apparatus 1. The input part 31 may also be used to define parameters for the control of the contrast agent injector.

### <Display>

The display 32 comprises any type of display, such as an LCD (Liquid Crystal Display) or CRT (Cathode Ray Tube) display, etc. The display 32 displays various X-ray CT medical images. For example, the screen in the display 32 displays tomographic images, volume rendering medical images, MPR medical images, etc. The display 32 may also display a viewing box compatible with MPR scanning.

The display 32 also displays the setup screen (not shown) for the scan conditions. The display 32 also displays the operating screen relating to scans by the gantry device 10. The display 32 displays the various parameters used in reconstruction. The display 32 also displays the setup screen for window level and window width. The display 32 may also be structured to display the setup screen used when the control parameters for the contrast agent injector are specified.

In the case of even at least in part for the input part 31 comprising GUI, it will also display the GUI. The display 32 may, for example, display the setup screen for parameters for scan conditions, reconstructing, medical image processing, etc. as GUI. The display 32 also displays the operating screen for the operation of the gantry and the bed as GUI.

### <Controller>

The condition setting part 33, the scan controller 34, the processor 35, the display controller 37 and the main controller 38 comprise processing equipment not shown such as, for example, a CPU (Central Processing Unit), a GPU (Graphic Processing Unit), or an ASIC (Application Specific Integrated Circuit), and a memory device not shown such as ROM (Read Only Memory), RAM (Random Access Memory) or a HDD (Hard Disc Drive). The control programs to perform the various functions of the equipment are stored in the memory device. A processing device such as a CPU carries out the function of respective parts by carrying out respective programs stored in the storing device.

### <Condition Setting Part>

The condition setting part 33 records various parameters such as the scan conditions, reconstruction conditions, window level or window width, input via the input part 31. The condition setting part 33 also records the screen data, etc., from setup screens for the various conditions noted above. Scan conditions include X-ray irradiation conditions, FOV (field of view), scope of scan, scan mode, slice thickness, etc.

X-ray irradiation conditions include parameters relating to the X-rays to which the subject is to be exposed. These parameters include, for example, in relation to the radiation X-rays, the tube current mA, the tube voltage kV, the rotation speed of the X-ray tube (rotational body 13), the interval between scans, etc. Parameters related to the field of view include control parameters for the operation of the X-ray diaphragm part 16, which is controlled by the gantry driving part 15. Reconstruction conditions include reconstruction functions, reconstruction intervals, etc. Scan modes can include, for example, the scanning method (conventional scan, helical scan, etc.). In the case of helical scans, it will include the helical pitch. Helical pitch can be altered corresponding to, for example, the conditions (operating speed, extent of movement, etc.) related to the operation of the bed board 23 by the bed operating part.

### <Scan Controller>

The scan controller 34 controls the various operations of X-ray scanning. The scan controller 34 receives the instruction to begin the scan from, for example, the input part 31 via the main controller 38. As a result of these instructions, the gantry device 10 begins the scan. In other words, the high voltage generator 14, the gantry driving part 15, the diaphragm driving part 17, the bed operating part 22, etc are controlled using the X-ray irradiation conditions, field of view, scope of scanning, scan mode and slice thickness, which are preset by the condition setting part 33.

For example, the scan controller 34 controls the bed operating part 22 based on the scope of X-ray scanning received from the condition setting part 33 via the main controller 38. As a result, the bed operating part 22 moves the bed apparatus 20 at the speed and to the extent specified. Furthermore, the scan controller 34 controls the high-voltage generator 14 based on the X-ray irradiation conditions received from the condition setting part 33. As a result, the high-voltage generator 14 controls the X-ray generating part 11 by, among other things, applying a high voltage to it. Furthermore, the scan controller 34 controls the gantry driving part 15 based on the conditions for rotation speed in the X-ray tube (rotational body 13) specified by the condition setting part 33. As a result, the gantry driving part 15 rotates the rotational body 13 at the designated speed. The scan controller 34 also controls the diaphragm driving part 17, based on the field of view specified by the condition setting part 33. As a result, the diaphragm driving part 17 moves the X-ray diaphragm part 16, controlling the scope of the irradiated X-rays. The scan controller 34 also controls the bed operating part 22, based on the scan mode specified by the condition setting part 33. As a result, the bed operating part 22 moves the bed apparatus 20 at the speed and to the extent specified.

### <Processor>

The processor 35 executes various processes with regard to the detection data sent from the gantry device 10 (data collector 18). The processor 35 comprises a preprocessor 35a, a reconstructing part 35b and an image generator 35c.

The preprocessor 35a executes preprocessing of the detection data detected using the gantry device 10 (X-ray detector 12), and produces projection data. Preprocessing involves logarithmic conversion processing, offset correction, sensitivity correction, beam hardening correction, etc.

The reconstructing part 35b reconstructs the projection data produced by the preprocessor 35a. This reconstruction is performed based on the reconstruction conditions received from the condition setting part 33 via the main controller 38. A method of choice such as, for example, the 2-dimensional Fourier conversion method or the convolution back projection method, etc., can be used to reconstruct tomographic image data. The volume data is produced by interpolating a plurality of reconstructed tomographic image data. The reconstruction of volume data can be used a method of choice such as the cone beam reconstruction method, the multi-slice reconstruction method or the enlargement reconstruction method, etc. When using multiple rows X-ray detection equipment, as described above, it is possible to reconstruct a broad scope of volume data based on data obtained from the volume scans.

The image generator 35c processes for the tomographic image data produced by the reconstructing part 35b or volume data to produce X-ray CT medical image data. For example, with volume data, it performs MPR (multi planar reconstructing), and rendering such as SR (surface rendering) or SSD (shaded surface Display), VR (volume rendering), MIP (maximum intensity prejection) or MinIP (minimum intensity prejection).

The image generator 35c also processes image data based on tomographic image data and volume data. Image processing involves, for example, sharpening the image, reducing or controlling noise, improving the S/N ratio, emphasizing contours, etc. Once this processing is completed, the image generator 35c creases the X-ray CT medical image data. The X-ray CT medical image data may be, for example, a contrast image showing the internal state of the subject before the injection of contrast agent. A contrast image showing the state of the subject after being injected with contrast agent is also an X-ray CT medical image. Other examples include images taken in preparation for scanning using the specifications for the scope of scanning the subject (hereinafter, sometimes referred to as a “scanogram”). It could also include a subtraction image between the non-contrast image and the contrast image. The contrast image or the subtraction image may also show highlighted contours of the region to which the contrast agent has been introduced, obtained by the image processing methods described above. Alternatively the contours may be extracted by threshold processing, based on CT numbers that show the contrast agent. The non-contrast image is one type of “basic image.” Other examples of basic images, however, include images taken after the injection of contrast agent. Hereinafter, the phrase “non-contrast image” does not only refer to a medical image of the subject before he/she is injected with contrast agent, but also includes images taken after the start of the administration of contrast agent (immediately after administration begins, etc.) In other words, an image that shows the designated area (bones etc.) of the subject that is under observation is equivalent to a basic image. The designated area, which is subject to observation, is equivalent to the area surrounding the area being highlighted using contrast agent.

The scanogram is a medical image based on tomographic image data of the subject scanned using a single X-ray projection angle. The scanogram is displayed on the display 32. In some cases, the user may setup the scan position of the SURE Start scan and the main scan based on the scanogram. The subtraction image is produced by the image generator 35c by subtraction processing of a basic image such as the non-contrast image with the contrast image. The following is an explanation of subtraction processing carried out by the image generator 35c. A SURE Start scan is an example of the first scan. The main scan is an example of the second scan.

The image generator 35c subtracts basic image data such as that of the non-contrast image from the contrast image data stored in the memory 36. The contrast image is an image based on collected data obtained from repeated (continual or intermittent) scanning by the gantry device 10. The non-contrast image is based on collected data obtained by either repeated or single scanning. These images are stored in the memory 36. The image generator 35c subtracts the basic image data collected by single scans from the contrast image data from the 1^{st} to the n^{th} scans. Moreover, the image generator 35c subtracts the basic image data collected by the correspondent interval scans from the contrast image data from the 1^{st} to the n^{th} scans. The following is an explanation of the examples above.

For example, the image generator 35c performs subtraction processing via a method of choice using a contrast image based on collected data from the 2^{nd} scan, and a basic image such as a non-contrast image, etc., read from its memory 36. The image generator 35c repeats the process sequentially for contrast images based on collected data from the 1^{st} to the n^{th} scans. As a result, subtraction images are produced that are compatible with the contrast images from the 1^{st} to the n^{th} scans.

Through subtraction processing of the contrast image and the basic image, subtraction images emphasize the scope of the entry of contrast agent in comparison with the internal structure of the subject, which is common to both images. As a result, it is easy to understand the scope of the entry of the contrast agent within the displayed medical image. In images by the SURE Start and main scans, for example, the parts that the user wishes to focus on are highlighted. As a result, they can be used to support the user in understanding when to switch over the scan, and see areas of pathological abnormalities. The X-ray CT medical image data produced by the image generator 35c in this way is stored sequentially in the memory 36 via the main controller 38.

In the embodiment described above, subtraction processing is carried out based on the reconstructed data. This is not always the case, however, and even if using pre-reconstruction projection data, it is possible to structure it such that subtraction processing is executed corresponding to the scan position. In such cases, the memory 36 stores, for example, the projection data obtained from the non-contrast scan. In this example, however, the projection data does not have to be that obtained from the non-contrast scan, it may also be that obtained from the scan taken after the contrast agent was injected into the subject (immediately after injection, etc.)

Furthermore, it is also possible for subtraction processing by the image generator 35c as described above to be performed using the produced contrast image and the contrast image produced at an earlier point (an example of the “second contrast image”). For example, the image generator 35c may perform subtraction using the contrast image formed in the scan immediately prior (the one before) and the presently formed contrast image. In this example, the image generator 35c performs subtraction processing between the contrast image from the 2^{nd} scan and the contrast image from the 1^{st} scan, wherein scans were taken n times. Furthermore, the image generator 35c performs subtraction processing between the contrast image from the 3^{rd} scan and the contrast image from the 2^{nd} scan. This sequential process is repeated, until subtraction processing is performed between the contrast image from the n^{th} scan and the contrast image from the n−1^{th} scan.

### <Memory and Display Controllers>

The memory 36 comprises semiconductor storage devices such as RAM or ROM. The memory 36 records detection data, projection data and X-ray CT medical image data. The display controller 37 performs various controls relating to image display. For example, based on display instructions for the aforementioned various types of X-ray CT medical image data, it receives the relevant image data from the memory 36 and displays it according to the specified format. In addition to this, the display controller 37 receives image data for the aforementioned various types of setup screen, and displays them on the display 32 according to the specified format. The display controller 27 also displays on the display 32 the changes over time in the contours of the region into which the contrast agent has been introduced on the medical images produced sequentially by the image generator 35c.

### <Main Controller>

The main controller 38 controls the operation of the gantry device 10, bed apparatus 20 and console apparatus 30, thereby performing overall control of the X-ray CT apparatus 1. For example, the main controller 38 controls the scan controller 34, instructing the gantry device 10 to perform either the preparatory scan or the main scan, and collect detection data. In addition, the main controller 38 controls the processor 35, instructing it to perform various types of processing (preprocessing, reconstruction, MPR processing, etc.) with regard to the detection data. Furthermore, by controlling the display controller 37, the main controller 38 displays X-ray CT medical images on the display 32 based on image data etc. stored in the memory.

### (Operation)

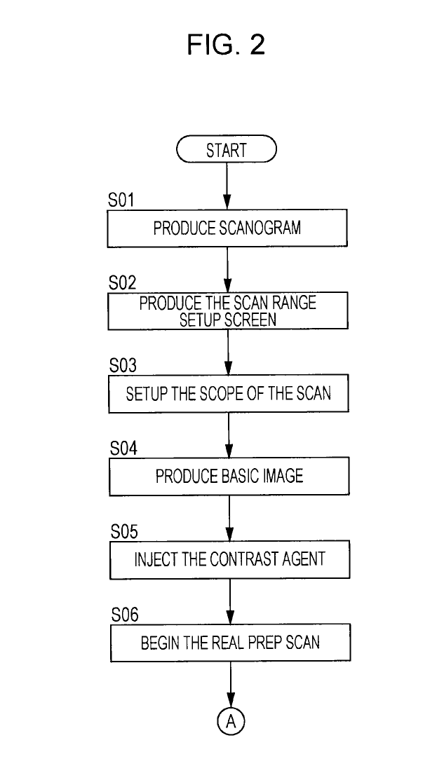

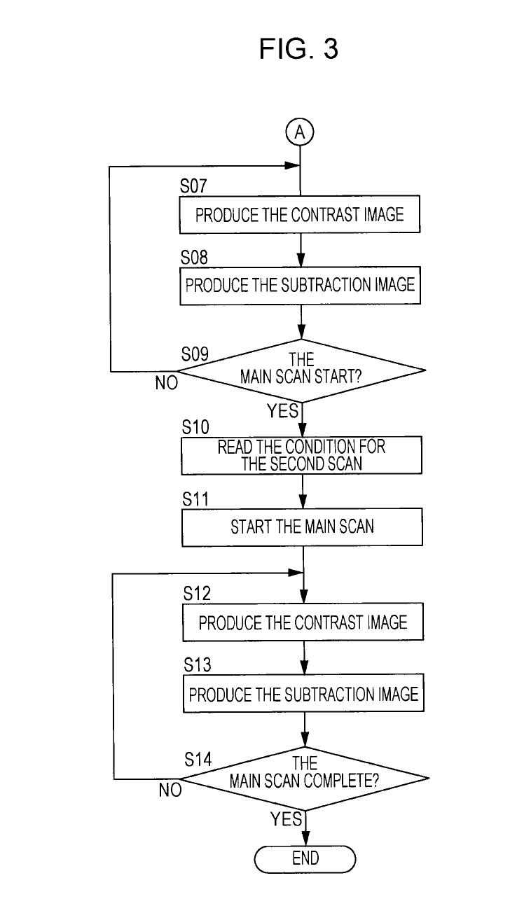

The following is an explanation of the operation of the X-ray CT apparatus 1 according to the present embodiment, in reference to FIGS. 2 and 3. The explanation will cover the process from the production of a scanogram, through to the production of medical images from the SURE Start scan and the main scan. This section explains cases where the non-contrast image is used as the basic image in order to perform subtraction processing.

### <S01>

When beginning a scan using contrast agent, the X-ray CT apparatus 1 forms a scanogram. In other words, the scan controller 34 takes a scanogram by controlling the high-voltage generator 14 on the gantry device 10, the gantry driving part 15, the diaphragm driving part 17 and the bed operating part 22, etc., based on pre-specified scan conditions. For example, the scan controller 34 may control the bed operating part 22 so as to move the relative positions of the bed board 23 and the gantry device 10, with the result that the subject is moved into the scanning position. Alternatively, the scan controller 34 controls the gantry driving part 15 and moves the rotational body 13. Additionally, the scan controller 34 controls the high-voltage generator 14, and scans the subject using a single X-ray projection angle. The data collector 18 collects detection data based on the X-rays that have passed through the subject. This collected data is sent to the console apparatus 30. The processor 35 produces a scanogram based on the collected data received by the console apparatus 30.

In this embodiment, the scan range setup screen is not necessarily based on the scanogram. For this reason, in some cases the process of producing a scanogram is omitted. Furthermore, the scanogram may be produced based on detection data collected when the non-contrast scan is taken in response to the main scan, and in such cases, the scan related to the production of the scanogram and the non-contrast scan in response to the main scan may be performed at the same time.

### <S02>

The display controller 37 produces the scan range setup screen based on the specified format received from the scanogram and the condition setting part 33, before displaying it on the display 32.

### <S03>

The scope of the SURE Start scan and the main scan are specified by the user, etc., via the input part 31, on the scan scope setup screen displayed on the display 32 in S02. In this way, setting up the scope of each scan on the scan scope setup screen ensures that the relevant scan scope is stored in the condition setting part 33 from the display controller 37 via the main controller 38.

### <S04>

Based on the specified scan conditions, the scan controller 34 controls the high-voltage generator 14 for the gantry device 10, the gantry driving part 15, the diaphragm 17 and the bed operating part 22, etc., and takes a scan to produce the non-contrast image. The non-contrast image may be produced in response to the SURE Start scan, or in response to the main scan. A non-contrast image for the SURE Start scan is produced under similar conditions (for example the conditions for the first scan) to the SURE Start scan, by scanning the subject before, for example, the contrast agent has been injected. A contrast image for the main scan is produced under the same scan conditions (for example the conditions for the second scan) as the main scan. The non-contrast image is different to the SURE Start scan and the main scan, however, in that in some cases it is produced based not on repeated scanning but on a single scan. Furthermore, the non-contrast image scan in this step (S04) is sometimes performed at different timing. The non-contrast image, for example, may be performed in the time between the injection of the contrast agent in the next step (S05) and the beginning of the SURE Start scan (S06).

### <S05>

Instruction is given from the input part 31 of the console apparatus 30 or from another operating part to inject the subject with the contrast agent using the contrast agent injector. If the scan taken to produce the non-contrast image in S04 is performed between S05 and S06, it may be subjected to perfusion analysis.

### <S06>

When the instruction is given via the input part 31 of the console apparatus 30 to begin the SURE Start scan, the scan controller 34 controls the start of the SURE Start scan based on the specified conditions (for example the conditions for the first scan). The bed operating part 22 may, for example, move the subject to the specified scanning position. Furthermore, the gantry driving part 15 rotates the X-ray tube, etc., by rotating the rotational body 13. The high-voltage generator 14 is used to repeatedly scan the subject under the specified X-ray irradiation conditions. The data collector 18 collects data based on the X-rays that pass through the subject. In the present embodiment, detection data is collected for a single rotation. This collected data is sent to the console apparatus 30.

### <S07>

In S06, the collected data collected from the SURE Start scan is sent to the console apparatus 30. The console apparatus 30 produces a contrast image using the processor 35.

### <S08>

Once the processor 35 produces the contrast image in S07, The non-contrast image corresponding to the SURE Start scan is read from the memory 36. The image generator 35c performs subtraction processing on this non-contrast image and contrast image. The result of this subtraction processing is the production of a subtraction image. The subtraction image is displayed on the display 32 via the display controller 37.

The display of the subtraction image makes it possible for the user to be supported in deciding when to start the main scan. In other words, even in areas where it is difficult to ascertain the state of flow of contrast agent, since the area where the contrast agent has been introduced is highlighted in the displayed image, the user can refer to the subtraction image and easily make a decision regarding the timing of the main scan.

### <S09>

The main controller 38 of the gantry device 10 judges whether or not there has been an instruction to switch over to the main scan via the input part 31. At the point at which such instructions have not yet been input, the main controller 38 (S09: No) repeats the processes S07 to S09 under, for example, the conditions for the SURE Start scan (conditions for the first scan, etc.) specified in the condition setting part 33. In other words, the X-ray CT apparatus 1 continues with the SURE Start scan. The “conditions for the first scan” may include not only scan conditions, but also a minimum of one of the other conditions relating to the production of X-ray CT medical image data including reconstruction conditions, the scope of the scan, the scan mode, etc.

### <S10>

The main controller 38 receives instructions via the input part 31 to switch over to the main scan (S09: Yes), and reads the conditions for the second scan from the condition setting part 33. The main controller 38 sends the conditions for the second scan to the scan controller 34. The conditions for the second scan equate, for example, to the tube current (mA) relating to X-ray irradiation and the irradiation interval (the operation of X-ray generating part 11), as well as the scope of irradiation (the range of opening or closing of the X-ray diaphragm 16), etc. The “conditions for the second scan” may include not only scan conditions, but also a minimum of one of the other conditions relating to the production of X-ray CT medical image data including reconstruction conditions, the scope of the scan, the scan mode, etc.

### <S11>

The scan controller 34 begins the main scan, based on the conditions for the second scan received from the main controller 38. Scanning conditions change during the main scan. For example, there may be changes to the tube current mA, the irradiation interval, or the scope of irradiation, etc. As a result, the main scan is begun under the conditions for the second scan, under the control of the scan controller 34.

### <S12>

During S11, the processor 35 produces a contrast image based on the collected data received from the console apparatus 30 as a result of the main scan.

### <S13>

The processor 35 produces a contrast image during S12, and reads the non-contrast image of the main scan from the memory 36. The image generator 35c performs subtraction processing of the non-contrast image and the contrast image. This process produces a subtraction image. The subtraction image is displayed via the display controller 37 on the display 32.

### <S14>

The main controller 38 of the gantry device 10 judges whether instruction has been given via the input part 31 to complete the main scan. At the point at which such instructions have not yet been input, the main controller 38 (S14: No) repeats the processes S12 to S14 under, for example, the conditions for the main scan (conditions for the second scan, etc.) specified in the condition setting part 33. In other words, the X-ray CT apparatus 1 continues with the main scan. The main controller 38 completes the scan when it judges an instruction has been given to complete the main scan (S14: Yes).

### (Function and Effect)

The following is an explanation of the functions and effects of the X-ray CT apparatus 1 according to Embodiment 1, as detailed above.

The X-ray CT apparatus 1 according to the present embodiment produces a subtraction image through subtraction processing of the contrast image and basic image during both the SURE Start scan and the main scan. As a result, the scope of flow of the contrast agent is highlighted relative to the internal structure of the subject, which is common to both the contrast image and the basic image. It is therefore easy to ascertain the scope of flow of the contrast agent from the displayed medical image. For example, in the SURE Start scan and the main scan, the area that the user wishes to focus on is highlighted. As a result, the user can be supported in ascertaining the timing of the switchover between scans, and any pathological abnormality, which can prevent mistakes in scanning when forming X-ray CT medical images using contrast agent.

### Second Embodiment

Next, there follows an explanation of the X-ray CT apparatus 1 according to Embodiment 2. Compared to Embodiment 1, Embodiment 2 features a different structure, etc., to the main controller 38 and the processor 35. The operation and processing details of other parts may also be different in response to this variance. Other than this, all parts are similar to those comprising the X-ray CT apparatus 1 according to Embodiment 1. The following is mainly an explanation of the differences between Embodiment 2 and Embodiment 1.

### (Outline)

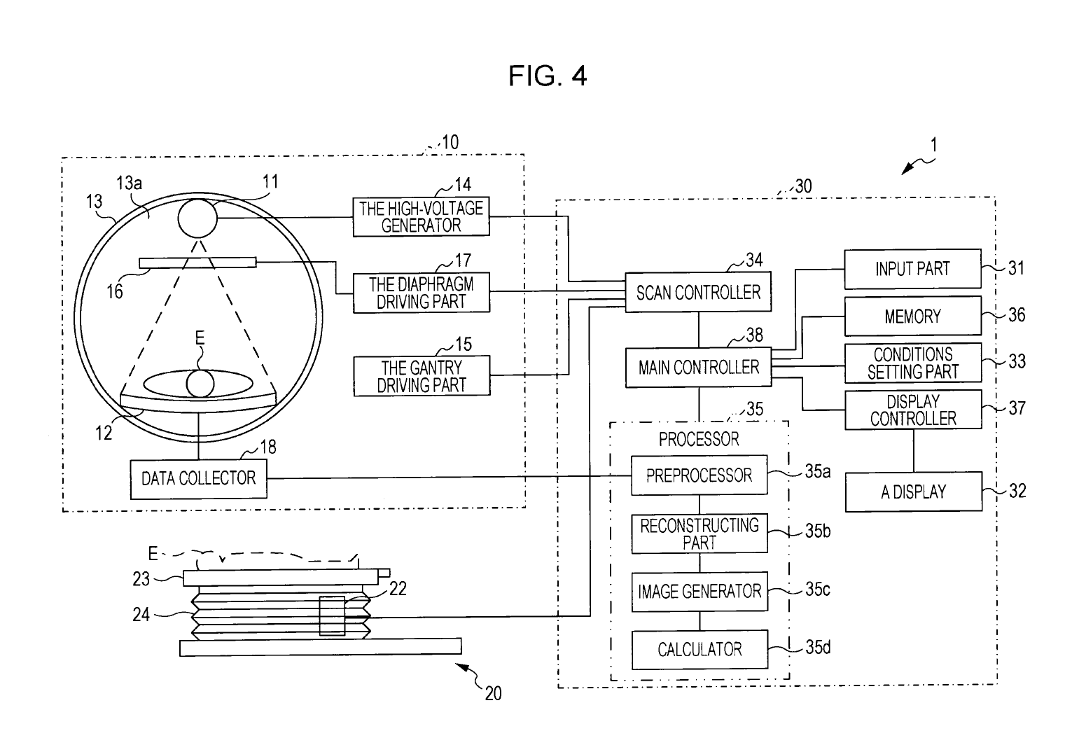

Similarly to that in Embodiment 1, the X-ray CT apparatus 1 according to Embodiment 2 also operates the gantry device 10 using various controllers on the console apparatus 30. In Embodiment 2, too, a subtraction image is produced based on the SURE Start scan. In Embodiment 2, however, the subtraction image based on the SURE Start scan is produced sequentially, and the surface area of the region indicated by the contrast agent is calculated on each image. Furthermore, the specified control is performed in relation to the production of the X-ray CT medical image at the point where the maximum surface area is calculated as the surface area is calculated sequentially. The specified control is, for example, the instruction to switch over to the main scan. The X-ray CT apparatus 1 includes a calculator 35d on the processor 35, as shown in FIG. 4, in order to carry out this operation.

### <Calculator>

As shown in FIG. 4, the processor 35 includes a calculator 35d, which receives image data for the subtraction image produced by the image generator 35c based on the SURE Start scan. Additionally, the calculator 35d prerecords the specified value α for the CT number (HU: Haunsfield units) or the pixel value shown by the contrast agent in the X-ray CT medical image. The specified value α may take the form of parameters for the specified scope. The calculator 35d compares the specified value α to each CT number or pixel value in the subtraction image data.

In this way, the calculator 35d identifies the part having CT number shown by the contrast agent from the pixels of the subtraction image based on the SURE Start scan, thus identifying the extent to which the contrast agent has been introduced. The calculator 35d calculates the surface area of the identified range, thereby calculating the surface area of the area into which the contrast agent has been introduced in the subtraction image.

The calculator 35d, for example, calculates the surface area of the area into which the contrast agent has been introduced within the image from the number of pixels in the image. Additionally, the calculator 35d may also replace the surface of the bed board 23 shown in the image based on the image scale with its actual size. In this way, the calculator 35d sequentially calculates the surface area of the relevant area of the sequentially formed subtraction image. For example, in a situation where the surface area is increasing, the calculator 35d does not reach a conclusion regarding the maximum surface area. In this case, the calculator 35d judges that the scope of flow of the contrast agent in the subtraction image has reached its maximum surface area at the point where any increase to the surface area ceases, or when said surface area begins to reduce.

When the calculator 35d judges that the scope of flow of the contrast agent has reached its maximum surface area, it sends an instruction to the controller 38 to stop the SURE Start scan. The main controller 38 stops the SURE Start scan at the gantry device 10 based on these instructions. It stops, for example, the operation of the X-ray generating part 11, the gantry driving part 15, and the diaphragm driving part 17.

### (Operation)

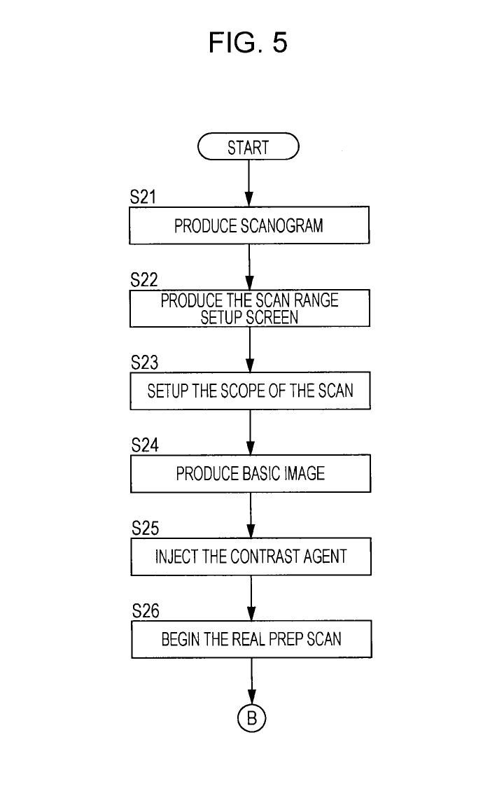

Next, there follows an explanation of the operation of the X-ray CT apparatus 1 according to the present Embodiment, in reference to FIGS. 5 to 7. This is mainly an explanation of the operation of the calculator 35d, and of the control based on said operation.

### <S21 to S28>

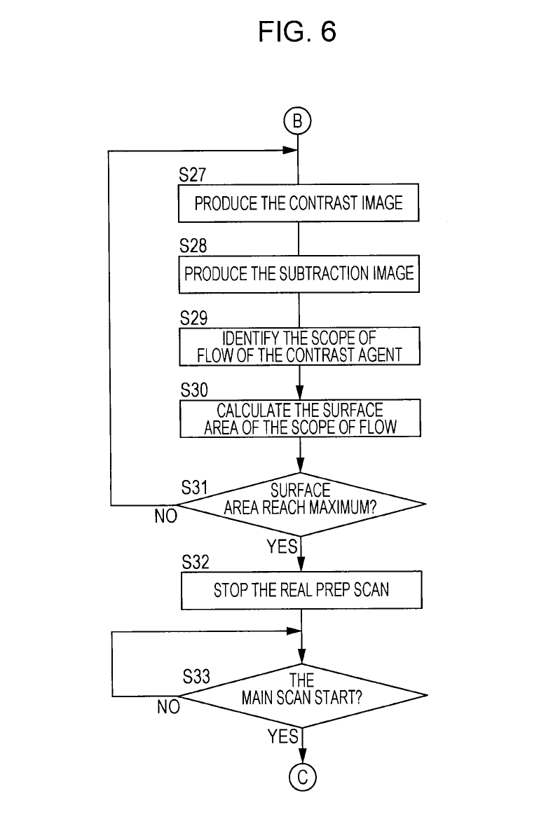

The production of the scanogram (S21), the production of the scan scope setup screen (22), the setup of the scan scope (S23), the production of the non-contrast image (S24), the injection of the contrast agent (S25) and the start of the SURE Start scan (S26) are similar to those performed in Embodiment 1 (S01 to S06) and so are omitted from this explanation. Furthermore, the formation of the contrast image based on the SURE Start scan (S27) and the production of the subtraction image (S28) shown in FIG. 6 are similar to those performed in Embodiment 1 (S07 and S08), and so are omitted from this explanation.

### <S29>

The calculator 35d compares the CT number of each pixel in the subtraction image formed sequentially based on the SURE Start scan with the pre-specified value α, in order to identify the scope of flow of the contrast agent.

### <S30>

The calculator 35d calculates the surface area of the scope of flow of the contrast agent, which was identified in the image.

### <S31>

The calculator 35d judges when the scope of flow of the contrast agent reaches its maximum surface area. For example, it may make this decision based on a comparison of said surface area with the surface area in the image taken one frame earlier. The processor 35 repeats the operations S27 to S31 until the calculator 35d calculates the maximum value (while S31: No).

### <S32>

When the calculator 35d judges that it has calculated the maximum surface area (S31: Yes), the calculator 35d sends an instruction to the main controller 38 to stop the SURE Start scan. The main controller 38 stops the SURE Start scan at the gantry device 10 via the scan controller 34.

### <S33˜S38>

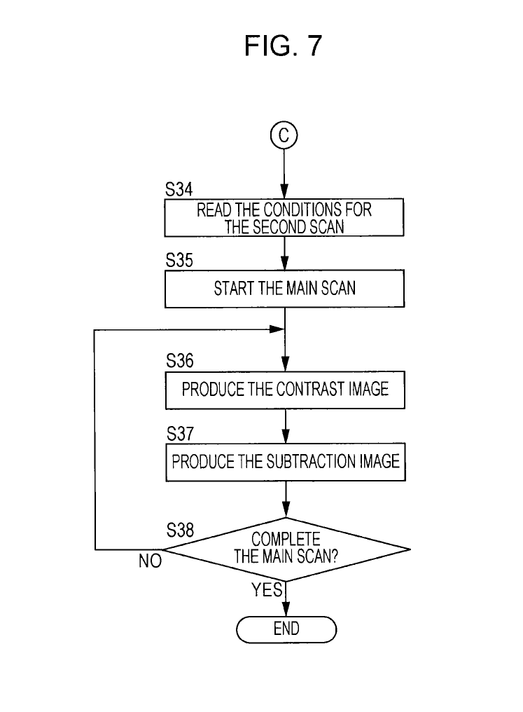

In FIGS. 6 and 7, the decision to begin the main scan (S33), the reading of the conditions for the second scan (S34), the start of the main scan (S35), the production of the contrast image (S36), the production of the subtraction image for the main scan (S37) and the decision to complete the main scan (S38) are similar to those performed in Embodiment 1 (S09 to S14) and so are omitted from this explanation. The main controller 38, etc., however, may be structured so as to start the main scan in response to the stopping of the SURE Start scan in S32.

### (Function and Effect)

The following is an explanation of the functions and effects of the X-ray CT apparatus 1 according to Embodiment 2, as detailed above.

The X-ray CT apparatus 1 according to the present embodiment produces a subtraction image through subtraction processing of the contrast image and basic image during both the SURE Start scan and the main scan. As a result, the scope of flow of the contrast agent is highlighted relative to the internal structure of the subject, which is common to both the contrast image and the basic image. It is therefore easy to ascertain the scope of flow of the contrast agent from the displayed medical image. For example, in the SURE Start scan and the main scan, the area that the user wishes to focus on is highlighted. As a result, the user can be supported in ascertaining the timing of the switchover between scans, and any pathological abnormality, which can prevent mistakes in scanning when forming X-ray CT medical images using contrast agent.

In addition to this, the X-ray CT apparatus according to Embodiment 2 calculates the surface area of the scope of flow of the contrast agent in the subtraction image based on the SURE Start scan, and judges when this surface area reaches its maximum value. Additionally, the main controller etc. of the X-ray CT apparatus 1 stop the SURE Start scan based on this judgment. This facilitates a reduction in the operating burden on the operator of the X-ray CT apparatus 1.

### [First Modification]

The following is an explanation of Modification 1 of the X-ray CT apparatus 1 according to Embodiment 2. The X-ray CT apparatus 1 according to Embodiment 2 stops the SURE Start scan in response to the decision by the calculator 35d that the surface area of the scope of flow of the contrast agent has reached its maximum value. It is not, however, restricted to this structure. For example, in response to the to the decision by the calculator 35d that it has calculated the maximum value, the main controller 38 may be input with instructions to stop the SURE Start scan (S32) and switch over to the main scan (S33). In this structure, after the instructions to switch over to the main scan (S33) have been input to the main controller 38, the operation of the X-ray CT apparatus 1 is similar to that of Embodiment 1.

Modification 1 can obtain similar effects to Embodiment 2. It facilitates further reductions in the operating burden on the operator of the X-ray CT apparatus 1.

### [Second Modification]

Next, there follows an explanation of Modification 2 of the X-ray CT apparatus 1 according to Embodiment 2. In the X-ray CT apparatus 1 according to Embodiment 2, the calculator 35d is structured to calculate the surface area of the scope of flow of the contrast agent using the subtraction image based on the SURE Start scan. It is not, however, restricted to this structure. The calculator 35d may, for example, calculate the surface area of the scope of flow of the contrast agent using the contrast image based on the SURE Start scan. Using this structure may in some cases allow the calculation of surface area by the calculator 35d to be done simultaneously with the subtraction processing by the image generator 35c. In the case of this type of structure, there may be some cases in which subtraction processing is not performed using the SURE Start scan.

### [Third Modification]

Next, there follows an explanation of Modification 3 of the X-ray CT apparatus 1 according to Embodiment 2. In the X-ray CT apparatus 1 according to Embodiment 2, the calculator 35d is structured to compare the CT number of each pixel to the specified value α. It is not, however, restricted to this structure. The calculator 35d may, for example, compare the CT number not of each pixel in the image, but rather of pixels from a selected area of the image, to the specified value α. A specified range may be pre-specified as the target for comparison by calculator 35d, for example, an area from the center of the image to one edge. Alternatively, the operator may specify any range via the input part 31.

Modification 3 can obtain similar effects to Embodiment 2, and it is possible to reduce the operating burden in the calculator 35d.

### [Fourth Modification]

Next, there follows an explanation of Modification 4 of the X-ray CT apparatus 1 according to Embodiment 2. In the X-ray CT apparatus 1 according to Embodiment 2, the calculator 35d is structured to sequentially calculate the surface area of the scope of flow of the contrast agent. It is not, however, restricted to this structure. The calculator 35d may, for example, be structured so as to calculate the maximum value by calculating the average CT number of the pixels in the subtraction image based on the SURE Start scan. Alternatively, it may use other statistical values (standard deviation, etc.) instead of the average.

Modification 4 can obtain similar effects to Embodiment 2.

### [Fifth Modification]

Next, there follows an explanation of Modification 5 of the X-ray CT apparatus 1 according to Embodiment 2. In the X-ray CT apparatus 1 according to Embodiment 2, the calculator 35d is structured to sequentially calculate the surface area of the scope of flow of the contrast agent. It is not, however, restricted to this structure. Instead of the maximum surface area (or a maximum statistical value as described above (see fourth Modification), it is possible to calculate the rate of the scope of flow of the contrast agent held by the whole that is covered by the surface area, and structure the apparatus so that a control signal is sent to the main controller 38 when said proportion exceeds a threshold value. This is similar when the structure involves specifying a statistical threshold, as in Modification 4.

Modification 4 can obtain similar effects to Embodiment 2.

### Third Embodiment

The following is an explanation of the X-ray CT apparatus 1 according to Embodiment 3. In comparison with Embodiment 2, the structure of the processor 35 is different in Embodiment 3, mainly in terms of the calculator 35d. The operation and processing details of other parts may also be different in response to this variance. Other than this, all parts are similar to those comprising the X-ray CT apparatus 1 according to Embodiment 2. The following is mainly an explanation of the points that are different to Embodiment 2.

Similarly to that in Embodiment 2, the X-ray CT apparatus 1 according to Embodiment 3 also operates the gantry device 10 using various controllers on the console apparatus 30. In Embodiment 3, too, a subtraction image is produced based on the SURE Start scan. In Embodiment 3, however, the calculator 35d receives volume data from the reconstructing part 35b, and calculates the volume (following, volume is referred to as dimensions) of the scope of flow of the contrast agent from the volume data.

Similarly to that in Embodiment 2, the processor 35 includes a calculator 35d. According to Embodiment 3, when the reconstructing part 35b produces the volume data by reconstructing projection data based on the SURE Start scan, the calculator 35d receives this volume data. Additionally, the calculator 35d prerecords the specified value α for the CT number (HU: Haunsfield units) or the pixel value shown by the contrast agent in the X-ray CT medical image. The specified value a may take the form of parameters for the specified scope. The calculator 35d compares the specified value α to each CT number or voxel value in the volume data.

In this way, the calculator 35d identifies the part demonstrating CT number or voxel value shown by the contrast agent volume data, thus identifying the extent to which the contrast agent has been introduced. The calculator 35d calculates the dimensions of the identified range. Furthermore, the calculator 35d sequentially calculates the accumulation from the sequentially formed volume data. For example, in a situation where the dimensions are increasing, the calculator 35d does not reach a conclusion regarding the maximum surface dimensions. In this case, the calculator 35d judges that the scope of flow of the contrast agent in the volume data has reached its maximum surface dimensions at the point where any increase to the dimensions cease, or when said dimensions begin to reduce.

When the calculator 35d judges that the scope of flow of the contrast agent has reached its maximum surface dimensions, it sends an instruction to the controller 38 to stop the SURE Start scan. The main controller 38 stops the SURE Start scan at the gantry device 10 based on these instructions. It stops, for example, the operation of the X-ray generating part 11, the gantry driving part 15, and the diaphragm driving part 17. Furthermore, the apparatus may be structured so that the main scan is started in response to the main controller 38, etc., stopping the SURE Start scan.

### (Function and Effect)

The following is an explanation of the functions and effects of the X-ray CT apparatus 1 according to Embodiment 3, as detailed above.

The X-ray CT apparatus 1 according to the present embodiment produces a subtraction image through subtraction processing of the volume data which produces with the contrast agent flows and the basic volume data (non-contrast volume data, etc.) during both the SURE Start scan and the main scan. As a result, the scope of flow of the contrast agent is highlighted relative to the internal structure of the subject, which is common to both the contrast image and the basic image. It is therefore easy to ascertain the scope of flow of the contrast agent from the displayed medical image. For example, in the SURE Start scan and the main scan, the area that the user wishes to focus on is highlighted. As a result, the user can be supported in ascertaining the timing of the switchover between scans, and any pathological abnormality, which can prevent mistakes in scanning when forming X-ray CT medical images using contrast agent.

In addition to this, the X-ray CT apparatus according to Embodiment 3 calculates the dimensions of the scope of flow of the contrast agent in the volume data based on the SURE Start scan, and judges when these dimensions reach their maximum value. Additionally, the main controller etc. of the X-ray CT apparatus 1 stop the SURE Start scan based on this judgment. This facilitates a reduction in the operating burden on the operator of the X-ray CT apparatus 1.

If the words “surface area of the image” in Modifications 1, 3, 4 and 5 of Embodiment 2 are replaced with “dimensions of the volume data”, similar modifications become applicable in Embodiment 3.

### Fourth Embodiment

The following is an explanation of the X-ray CT apparatus 1 according to Embodiment 4. In comparison with Embodiments 2 and 3, the structure of the processor 35 is different in Embodiment 4, mainly in terms of the calculator 35d. The operation and processing details of other parts may also be different in response to this variance. Other than this, all parts are similar to those comprising the X-ray CT apparatus 1 according to Embodiments 2 or 3. The following is mainly an explanation of the points that are different to Embodiments 2 and 3.

Similarly to that in Embodiments 2 and 3, the X-ray CT apparatus 1 according to Embodiment 4 also operates the gantry device 10 using various controllers on the console apparatus 30, and produces a subtraction image based on the SURE Start scan. Furthermore, it is similar in that the calculator 35d calculates whether the maximum value (surface area or dimensions) of the scope of flow of the contrast agent has been reached from the image or the volume data. According to Embodiment 4, however, at the point at which the calculator 35d calculates the maximum value (surface area or dimensions) of the scope of flow of the contrast agent, the main controller 38 sends an instruction to stop reconstruction, making it different to the other embodiments. Alternatively, it differs from other embodiments in that, in response to the state of flow of the contrast agent, the calculator 35d sends instructions to change the reconstruction conditions.

When the calculator 35d calculates the maximum value (surface area or dimensions) of the scope of flow of the contrast agent, an instruction is sent to the main controller 38 to either change the reconstruction conditions, or stop the reconstruction. The main controller 38 receives this instruction and changes the processing being done by the reconstructing part 35b. The main controller 38 may, for example, change preset conditions related to the reconstruction interval or the reconstruction function by the reconstructing part 35b. Alternatively, the main controller 38 may stop the reconstruction by the reconstructing part on receiving the instruction. The main controller 38 may also change the preset conditions, such as the transition in calculated values for the scope of flow of the contrast agent, the transition in the proportion of the whole that is covered by said area, the reconstruction interval in response to transition in average CT numbers, or the reconstruction function.

### (Function and Effect)

The following is an explanation of the functions and effects of the X-ray CT apparatus 1 according to Embodiment 4, as detailed above.

The X-ray CT apparatus 1 according to the present embodiment produces a subtraction image through subtraction processing of the contrast image and basic image during both the SURE Start scan and the main scan. As a result, the scope of flow of the contrast agent is highlighted relative to the internal structure of the subject, which is common to both the contrast image and the basic image. It is therefore easy to ascertain the scope of flow of the contrast agent from the displayed medical image. For example, in the SURE Start scan and the main scan, the area that the user wishes to focus on is highlighted. As a result, the user can be supported in ascertaining the timing of the switchover between scans, and any pathological abnormality, which can prevent mistakes in scanning when forming X-ray CT medical images using contrast agent.

In addition to this, in the X-ray CT apparatus according to Embodiment 4, calculator 35d calculates whether the maximum value (surface area or dimensions) of the scope of flow of the contrast agent has been reached from the image or the volume data. Additionally, the main controller 38 receives the relevant instruction and stops the processing being done by the reconstructing part 35b. It may also change the preset conditions, such as the transition in calculated values for the scope of flow of the contrast agent, the transition in the proportion of the whole that is covered by said area, the reconstruction interval in response to transition in average CT numbers, or the reconstruction function. This may facilitate a reduction in the operating burden on the operator of the X-ray CT apparatus 1. It may also facilitate a reduction in the processing burden on the operator of console apparatus 30.

### Fifth Embodiment

The following is an explanation of the X-ray CT apparatus 1 according to Embodiment 5. In comparison with Embodiments 2 and 3, the structure of the main controller 38 is different in Embodiment 5. The operation and processing details of other parts may also be different in response to this variance. Other than this, all parts are similar to those comprising the X-ray CT apparatus 1 according to Embodiments 2 or 3. The following is mainly an explanation of the points that are different to Embodiments 2 and 3.

Similarly to that in Embodiments 2 and 3, the X-ray CT apparatus 1 according to Embodiment 5 also operates the gantry device 10 using various controllers on the console apparatus 30. In Embodiment 5, too, a subtraction image is produced based on the SURE Start scan. Furthermore, it is similar in that the calculator 35d calculates whether the maximum value (surface area or dimensions) of the scope of flow of the contrast agent has been reached from the image or the volume data. According to Embodiment 5, however, at the point at which the calculator 35d calculates the maximum value (surface area or dimensions) of the scope of flow of the contrast agent, supplementary information is added to the image data (dynamic image data), making it different from other embodiments.

When the calculator 35d calculates the maximum value (surface area or dimensions) of the scope of flow of the contrast agent, the main controller 38 may, for example, add information related to the point at which the maximum value was reached, in a format that can be referred to by the user. Specifically, information (text information, etc.) that communicates the fact that the maximum value has been reached is added to the image frame scanned at that point. In addition, the passage of time between the beginning of the scan and the calculation of the maximum value may be added to the produced dynamic image data.

This embodiment shares the aspects of the calculator 35d calculating the surface area or dimensions of the scope of flow of the contrast agent, but it is possible to use a structure in which the calculator 35d does not calculate whether or not the maximum value has been reached. The calculator 35d may also display the transition in calculated values for the scope of flow of the contrast agent, the transition in the proportion of the whole that is covered by said area or the transition in average CT numbers as a graph, etc.

### (Function and Effect)

The following is an explanation of the functions and effects of the X-ray CT apparatus 1 according to Embodiment 5, as detailed above.

The X-ray CT apparatus 1 according to the present embodiment produces a subtraction image through subtraction processing of the contrast image and basic image during both the SURE Start scan and the main scan. As a result, the scope of flow of the contrast agent is highlighted relative to the internal structure of the subject, which is common to both the contrast image and the basic image. It is therefore easy to ascertain the scope of flow of the contrast agent from the displayed medical image. For example, in the SURE Start scan and the main scan, the area that the user wishes to focus on is highlighted. As a result, the user can be supported in ascertaining the timing of the switchover between scans, and any pathological abnormality, which can prevent mistakes in scanning when forming X-ray CT medical images using contrast agent.

Furthermore, the X-ray CT apparatus 1 according to Embodiment 5 supplies values calculated by the calculator 35d as reference information for the user. This may facilitate a reduction in the operating burden on the operator of the X-ray CT apparatus 1.

### Sixth Embodiment

The following is an explanation of the X-ray CT apparatus 1 according to Embodiment 6. In comparison with Embodiments 2 and 3, the structure of the main controller 38 is different in Embodiment 5. The operation and processing details of other parts may also be different in response to this variance. Other than this, all parts are similar to those comprising the X-ray CT apparatus 1 according to Embodiments 2 or 3. The following is mainly an explanation of the points that are different to Embodiments 2 and 3.

According to Embodiment 6, in contrast to Embodiments 2 and 3, rather than controlling the apparatus to switch over from the SURE Start scan to the main scan it is controlled to switch to intermittent scanning.

For example, according to Embodiment 6, intermittent multiple scans of the subject are expected. When the main controller 38 begins the operation to start the scan via the input part, etc., it reads the scan conditions. According to Embodiment 6, if instructions are given via the input part 31 to perform intermittent multiple scans, the prerecorded settings for said intermittent scanning are read.

### <Image Generator/Calculator>

The image generator 35c forms a subtraction image based on the first scan. The calculator 35d prerecords the specified value α for the CT number or the pixel value shown by the contrast agent in the X-ray CT medical image or volume data. The specified value α may take the form of parameters for the specified scope. The calculator 35d receives the relevant image data from the image generator 35c, and compares the specified value α to the CT number or pixel (voxel) value for each pixel (voxel) in the subtraction image data.

In this way the calculator 35d identifies the part with CT number shown by the contrast agent, from the subtraction image pixels (voxels) based on the first scan, thus identifying the scope of flow of the contrast agent. The calculator 35d calculates the surface area or dimensions of the relevant identified scope, thus calculating the surface area or dimensions of the scope of flow of the contrast agent in the subtraction image.

When the calculator 35d judges that the scope of flow of the contrast agent in the image has reached its maximum surface area (dimensions), it sends instructions to the main controller 38 to stop the first scan. The main controller 38 stops the scan by the gantry device 10 based on said instructions.

Next, the main controller 38 begins the second scan under the conditions for intermittent scanning that were read as described above. For example, it may begin the second scan after a specified period of time has lapsed since stopping the first scan. The conditions for the second scan may be specified differently to those for the first scan. The definition of scan conditions is as explained above.

The modifications applied to Embodiment 2 may also be applied to Embodiment 6. It is also possible, when appropriate, to combine this embodiment with the other embodiments.

### (Function and Effect)

The following is an explanation of the functions and effects of the X-ray CT apparatus 1 according to Embodiment 6, as detailed above.

The X-ray CT apparatus 1 according to the present embodiment calculates the surface area or dimensions of the scope of flow of the contrast agent by subtraction processing during the first scan, and judges whether the maximum value of said surface area or dimensions has been reached. Additionally, the control part etc. of the X-ray CT apparatus 1 switches over from the first to the second scan based on said judgment. This facilitates a reduction in the operating burden on the operator of the X-ray CT apparatus 1.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus, comprising:
a collector, which scans the subject with X-rays and collects the data,
a memory, which prerecords a basic image, including the designated area of said subject,
a controller, which controls said collector so as to repeatedly perform the first scan, with the conditions for the first scan, of said designated part of said subject after administration of a contrast agent, and furthermore upon receipt of the input of a specified trigger, and to perform the second scan of said subject using the conditions for the second scan, and
an image generator, which sequentially generates the first medical image based on the repetitively-collected data as said first scan is being performed, and produces a subtraction image between said first medical image and said basic image, in addition to sequentially producing the second medical image based on collected data when said second scan is performed.

2. The X-ray CT apparatus according to claim 1, wherein
said first scan is the prep scan, while said second scan is the main scan.

3. The X-ray CT apparatus according to claim 1, further comprising:
a display that displays said subtraction image, and
an input part, for the input of said specified trigger.

4. The X-ray CT apparatus according to claim 1, further comprising:
a bed apparatus with a board on which said subject is positioned, and wherein,
said conditions for the first scan and said conditions for the second scan include a least one of condition relating to the irradiation conditions and scope of irradiation, as well as the displacement or otherwise of the relative location between said collector and said board.

5. The X-ray CT apparatus according to claim 1, wherein
a calculator, which calculates the part showed by said contrast agent, based on the CT number expressed by each pixel in said contrast image or said subtraction image generated by said image generator.

6. The X-ray CT apparatus according to claim 5, wherein
said calculator calculates the maximum value of the applicable area, and sequentially calculates the area of the part showed by said contrast agent.

7. The X-ray CT apparatus according to claim 2, wherein
said collector collects volume data on said subject by said main scan, and comprises a calculator which calculates the part showed by the contrast agent within said volume data.

8. The X-ray CT apparatus according to claim 7, wherein
said calculator sequentially calculates the volume of the part showed by said contrast agent to calculate the relevant maximum volume.

9. The X-ray CT apparatus according to either one of claim 6 or 8, wherein
said controller stops said prep scan by said collector in response to the calculating said maximum value by said calculator.

10. The X-ray CT apparatus according to either one of claim 6 or 8, characterized in that
said controller begins said main scan by said collector in response to the calculating said maximum value by said calculator.

11. The X-ray CT apparatus according to either one of claim 6 or 8, wherein
said image generator produces said contrast image and said basic image by reconstructing said data obtained from said collector, and
said controller stops said reconstructing by said image generator in response to the calculating said maximum value by said calculator.

12. The X-ray CT apparatus according to either one of claim 6 or 8, wherein
said image generator produces said contrast image and said basic image by reconstructing said data received from said collector, and
said controller increases the intervals in which said reconstructing performed by said image generator in response to the calculating said maximum value by said calculator.

13. The X-ray CT apparatus according to either one of claim 6 or 8, wherein
said image generator produces a motion image of said contrast image or said subtraction image based on said data received repeatedly from said collector, and
said controller adds the information indicating that the part showed by said contrast agent was the maximum as an additional information for the frame of the correspondent motion image, in response to the calculating said maximum value by said calculator.

14. The X-ray CT apparatus according to either one of claim 6 or 8, wherein
said image generator produces a motion image of said contrast image or said subtraction image based on said data received repeatedly from said collector, and
said controller adds the information indicating the numerical value of said maximum value as an additional information for the image data of motion images, in response to the calculating said maximum value by said calculator.

15. The X-ray CT apparatus according to either one of claim 6 or 8, wherein
said image generator produces a motion image of said contrast image or said subtraction image based on said data received repeatedly from said collector, and
said controller adds the information indicating the state of change to said area of the part showing said contrast agent, calculated by said calculator as an additional information.

16. The X-ray CT apparatus according to either one of claim 6 or 8, characterized in that
said image generator produces a motion image of said contrast image or said subtraction image based on said data received repeatedly from said collector, and
said controller adds the information indicating the state of change to said volume of the part showing said contrast agent, calculated by said calculator as an additional information.

17. The X-ray CT apparatus according to claim 1, wherein
said controller performs an intermittent scan by performing said first and second scans by said collector.

18. An medical image display method for X-ray CT apparatus, characterized by
a step wherein the subject is scanned by the collector prior to being injected with contrast agent, and basic image data is collected from this scan showing the internal state of the subject,
a step wherein, based on said basic image data, the basic image is produced by the image generator,
a step wherein the first scan of the subject, having been injected with contrast agent, is performed by said collector using repeated scanning, and contrast image data showing the internal state of the subject is collected from said first scan,
a step wherein said image generator sequentially produces a contrast image based on said contrast image data collected repeatedly in said first scan,
a step wherein said image generator sequentially produces a subtraction image from said contrast image and said basic image,
a step wherein said sequentially produced subtraction image is displayed,
a step wherein the specified trigger is input, and the apparatus switches over from the first to the second scan, using the conditions for the second scan, and
a step wherein the second medical image is produced based on the data collected in the second scan.
